(19) United States
(12) Patent Application Publication
(10) Pub. No.: US 2022/0167889 A1
(43) Pub. Date: Jun. 2, 2022

(54) APPARATUS AND METHOD FOR WRIST-WORN PULSE OXIMETRY

(71) Applicant: Detect Biosciences Inc., Cambridge, MA (US)

(72) Inventors: Eric Kriegstein, Boston, MA (US); Michael Bernstein, San Ramon, CA (US)

(73) Assignee: Detect Biosciences Inc., Cambridge, MA (US)

(21) Appl. No.: 17/247,140

(22) Filed: Dec. 1, 2020

(51) Int. Cl. A61B 5/1455; A61B 5/00

(52) U.S. Cl. CPC A61B 5/14552; A61B 5/6831; A61B 5/681; A61B 5/0017

(57) ABSTRACT

An apparatus and method for appendage worn biometric sensing devices. A moveable sensor carrier is captively retained in a housing containing the biometric device. A biasing component urges the moveable sensor carrier in contact with a skin surface of a monitored subject to maintain a regulated pressure of the movable carrier against the skin surface. A biometric sensor carried in the movable carrier enjoys an improved signal quality regardless of the exact positioning of the biometric sensor by the user.

### BACKGROUND OF THE INVENTION

The present invention relates to pulse oximetry, and more particularly to carriers for pulse oximetry devices to couple the sensing device to a patient.

With pulse oximetry devices, an optical sensor is pressed against the subject's tissue to measure peripheral capillary oxygen saturation (SPO2) levels of the subject. The performance of the devices is strongly dependent on how hard they are pressed on the subject's tissue.

Existing pulse oximetry devices to obtain SPO2 from a wrist worn pulse oximeter use tension on a wristband to force contact of the optical sensors into the skin. Most pulse oximeters of this type require careful placement and precise tightening of the wristband to function properly. However, most user's requiring monitoring of by a pulse oximeter just want to easily apply the device and continue with their normal activities.

In this type of device, if the optical sensors are not tightly against the skin then light leaks past and does not pass through tissue. If the optical sensors are pressed too hard into the underly peripheral tissues, the blood is forced out of the tissue and the signal becomes weaker. Thus, it is difficult to obtain the proper pressure on a patient's wrist tissue to optimize the optical signal quality for obtaining SPO2 values without reducing comfort and ease of application.

As can be seen, there is a need for an improved apparatus and methods for wrist-worn pulse oximeter devices.

### SUMMARY OF THE INVENTION

In one aspect of the present invention, a carrier for a biometric sensing device is disclosed. The carrier includes a housing having at least one sidewall defining an interior cavity. A proximal surface is oriented towards a monitored subject, when the carrier is applied to the monitored subject. A distal surface is oriented away from the monitored subject. A carrier opening is defined in the proximal surface. A moveable sensor carrier has a proximal face and an interior space dimensioned to carry one or more biometric sensors therein. A lip protrudes around a distal end of the carrier. The lip is dimensioned to captively retain the carrier within the carrier opening. A biasing element is configured to urge the moveable sensor carrier so that it protrudes outwardly from the proximal surface of the housing.

In some embodiments, a plurality of apertures extend between the interior space and the proximal face. The plurality of apertures is adapted to receive at least one of the one or more biometric sensors to position the at least one of one or more biometric sensors in contact with a skin surface of the monitored subject when the carrier is applied to the monitored subject.

In some embodiments, the at least one biometric sensor is an optical transmitter and an optical receiver.

In some embodiments, a seal is disposed about the moveable sensor carrier. The seal is configured to seal the carrier opening to prevent infiltration of a contaminant into the interior cavity.

In some embodiments, a connector configured to couple the housing to a band dimensioned to be worn about an appendage of the monitored subject. The band may be a wrist band.

In some embodiments, the connector is an elongate slot defined along a side edge of the housing. The elongate slot is configured to threadingly receive the band.

In some embodiments, the biasing element is a spring received within the housing and configured to urge the carrier outwardly from the interior cavity. The biasing element may have a selected tension such that when the housing is positioned against a skin surface of the monitored subject's wrist, the moveable sensor carrier is in a partially retracted condition within the housing.

In other embodiments, the moveable sensor carrier may reciprocate within the carrier opening to maintain a regulated pressure of the proximal face of the moveable sensor carrier in contact with the skin surface of the monitored subject.

These and other features, aspects and advantages of the present invention will become better understood with reference to the following drawings, description and claims.

### BRIEF DESCRIPTION OF THE DRAWINGS

FIG. 1 is a perspective view of the invention, shown in use;

FIG. 2 is a perspective view of the invention;

FIG. 3 is an exploded view of the invention;

FIG. 4 is a section view of the invention, taken along line 4-4 in FIG. 2;

FIG. 5 is a section view of the invention, taken along line 5-5 in FIG. 4; and

FIG. 6 is a section view of the invention, taken along line 6-6 in FIG. 1, illustrating the pressure spring force 30 creates on carrier 16 against user 34's skin.

### DETAILED DESCRIPTION

The following detailed description is of the best currently contemplated modes of carrying out exemplary embodiments of the invention. The description is not to be taken in a limiting sense, but is made merely for the purpose of illustrating the general principles of the invention.

Broadly, embodiments of the present invention provide improved apparatus and methods for wrist-worn pulse oximetry. The present invention controls the pressure of an optical sensor of a pulse oximetry device against the skin, regardless of the exact positioning of the optical sensor by the user. The improved design can improve the reliability and accuracy of the signal monitoring. It can also improve the user experience by making the application of the pulse oximetry device easier and make wearing the pulse oximeter more comfortable.

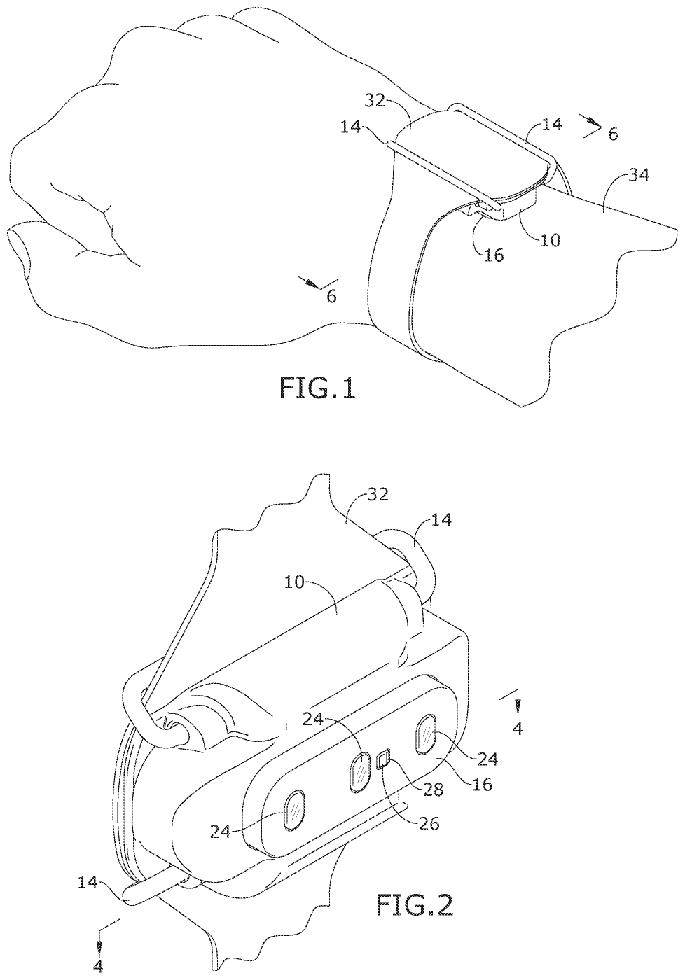

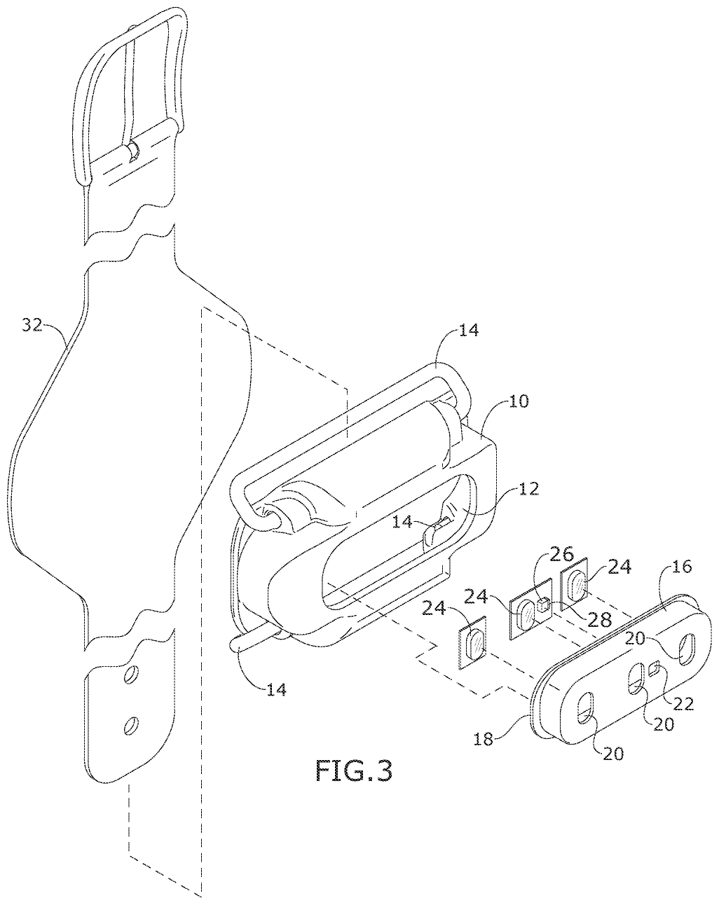

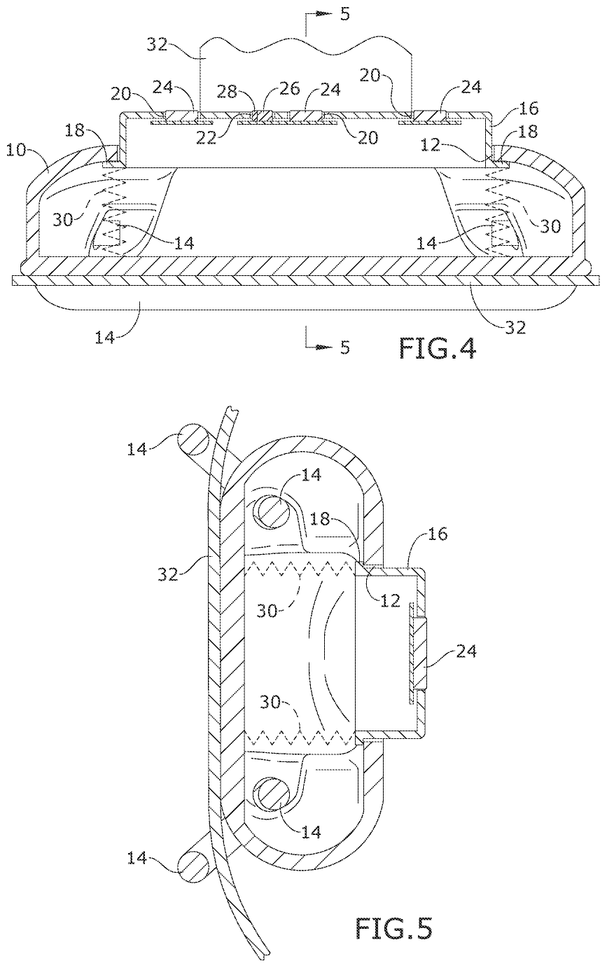

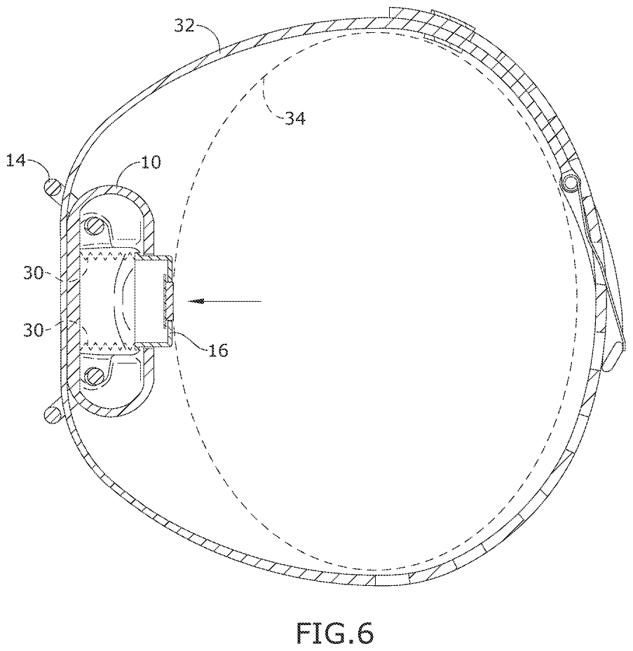

As seen in reference to FIGS. 1-6, a pulse oximeter device is shown. The pulse oximeter includes housing 10. The housing 10 has a connector configured for coupling the device to a wristband 32 for wear of the pulse oximeter to a wearer's wrist. In the non-limiting embodiment shown a pair of pair of rings 14, defining a coupling slot, are provided through which the wristband 32 is threaded to couple the pulse oximeter device to the wristband 32. The wristband 32 is dimensioned to encircle a wearer's wrist 34 and has a buckle or other suitable coupling to adjust the wristband 32 about the wearer's wrist 34.

The housing 10 is defined by at least one sidewall having a proximal face positioned in contact with the wearer's wrist 34, when applied, and a distal face positioned away from the wearer's wrist, when applied. The housing 10 has an interior cavity for receiving a pulse oximetry sensor.

A carrier opening 12 is defined in the proximal face of the housing 10. The carrier opening 12 is dimensioned to receive a moveable sensor carrier 16. The movable sensor carrier 10 has a retaining lip 18 protruding from a distal end of the carrier and a sensor face oriented with the proximal face to face the wearer's skin. A plurality of apertures 20 are defined in the sensor face for communication of an optical signal with each of an optical transmitter 24 and an optical receiver 26 of the pulse oximetry sensor. Optionally, a temperature aperture 22 may be provided for communication with a temperature sensor 28 mounted on the moveable sensor carrier.

The optical transmitter 24, optical receiver 26, and temperature sensor 28 protrude through the respective plurality of apertures 20 and 26 to position the respective sensors in contact with a skin surface of the wearer. The optical transmitter 24 may be selected from the group consisting of light emitting diodes (LEDs), laser diodes, incandescent lights, and florescent lights.

A biasing component 30, such as a spring, electro-mechanical drive, pneumatics or hydraulics, is carried within the housing 10 and is configured to urge the movable sensor carrier 10 so that it protrudes outwardly from the housing 10 while being captively retained within the carrier opening 12 by the retaining lip 18. The biasing component 30 has a selected tension such that when the housing 10 is positioned against the user's wrist 10, the movable sensor carrier 16 is in a partially retracted condition within the housing. The selected tension of the biasing component 30 is chosen so that as the user is engaged in activities, the moveable carrier 16 may reciprocate within the housing 10 to maintain a constant pressure of the one or more optical sensors in contact with the user's skin.

The housing 10 and the moveable carrier 16 can be made from metal or plastic. The moveable carrier 16 may be connected to the housing 10 with a flexible seal to prevent the infiltration of dirt, water, and other contaminants from entering the device.

An electronics circuit may be carried within the housing 10 is configured to perform a desired biometric function, such as pulse oximetry. In some embodiments, the electronics circuit could function with as few sensor components as a red led, an infrared LED and a photodetector. Alternatively, the sensor components may include a green led and photodetector to produce an app such as that used for heartrate. The signal can also be used for a blood pressure measurement. The optional temperature sensor can determine if the wearer has a fever or is otherwise at a desired thermal condition. Other optional sensor components may include surface electrodes that can be configured for measurement of a skin resistance or may be incorporated as a sensor of an EKG monitor.

The electronics circuit may be connected to the sensor components carried on the movable carrier 16 with a flex circuit, a flexible wire, resilient contactors, or a combination. In some embodiments, the pressure of the biasing component 30 exerted on the movable carrier 16 can be controlled electronically such that a feedback loop can be used to optimize the pressure to obtain the best possible signal from the one or more sensors. The electronics circuit may include a display for a processed biometric signal sensed by the biometric sensors. The electronics circuit may also include a communications element for communication with a mobile computing device.

As will be appreciated, the present invention provides an apparatus and method to enhance the performance characteristics of any wearable biosensor dependent on reliable positioning of the biosensor in proximity or in proper contact with the wearer to provide the best performance. By implementing this in a biosensing device, the device is more accurate, reliable and comfortable for the user. The present invention is applicable in the athletics, home health, and hospital applications where movements of the patient or subject are encountered. The invention is suitable for monitoring neonates who move a lot. Likewise, the present invention may be applicable in veterinary care settings.

It should be understood, of course, that the foregoing relates to exemplary embodiments of the invention and that modifications may be made without departing from the spirit and scope of the invention as set forth in the following claims.

What is claimed is:

1. A carrier for a biometric sensing device, comprising:
a housing having at least one sidewall defining an interior cavity, a proximal surface oriented towards a monitored subject, when applied to the monitored subject, and a distal surface oriented away from the monitored subject, and a carrier opening defined in the proximal surface;
a moveable sensor carrier having a proximal face and an interior space dimensioned to carry one or more biometric sensors therein, and a lip protruding around a distal end of the carrier, the lip dimensioned to captively retain the carrier within the carrier opening; and
a biasing element configured to urge the moveable sensor carrier so that it protrudes outwardly from the proximal surface of the housing.

2. The carrier of claim 1, further comprising:
a plurality of apertures extending between the interior space and the proximal face, the plurality of apertures adapted to receive at least one of the one or more biometric sensors, to position the at least one of one or more biometric sensors in contact with a skin surface of the monitored subject, when the carrier is applied to the monitored subject.

3. The carrier of claim 2, wherein at least one biometric sensor comprises:
an optical transmitter; and
an optical receiver.

4. The carrier of claim 1, further comprising:
a seal disposed about the moveable sensor carrier configured to seal the carrier opening to prevent infiltration of a contaminant into the interior cavity.

5. The carrier of claim 1, further comprising:
a connector configured to coupling the housing to a band dimensioned to be worn about an appendage of the monitored subject.

6. The carrier of claim 5, wherein the band is a wrist band.

7. The carrier of claim 5, wherein the connector is an elongate slot defined along a side edge of the housing, the elongate slot configured to threadingly receive the band.

8. The carrier of claim 1, wherein the biasing element is a spring received within the housing and configured to urge the carrier outwardly from the interior cavity.

9. The carrier of claim 8, wherein the biasing element has a selected tension such that when the housing is positioned against a skin surface of the monitored subjects wrist, the moveable sensor carrier is in a partially retracted condition within the housing.

10. The carrier of claim 9, wherein the moveable sensor carrier may reciprocate within the carrier opening to maintain a constant pressure of the proximal face of the moveable sensor carrier in contact with the skin surface of the monitored subject.
